(19) United States
(12) Patent Application Publication
(10) Pub. No.: US 2015/0126803 A1
(43) Pub. Date: May 7, 2015

(54) WOMB MIMICKING INCUBATOR

(71) Applicant: ASPECT IMAGING LTD., Shoham (IL)

(72) Inventor: Uri RAPOPORT, Moshav Ben Shemen (IL)

(21) Appl. No.: 14/339,783

(22) Filed: Jul. 24, 2014

Related U.S. Application Data
(60) Provisional application No. 61/899,909, filed on Nov. 5, 2013.

(51) Int. Cl. A61G 11/00; A61M 21/02; A61B 5/021; A61B 5/00; A61B 5/0205; A61M 21/00; A61B 5/16

(52) U.S. Cl. CPC A61G 11/008; A61M 21/0094; A61M 21/02; A61B 5/165; A61B 5/4836; A61B 5/0205; A61B 5/021; A61B 5/4266; A61B 5/486; A61G 2200/14; A61M 2021/0044; A61M 2021/0066; A61M 2021/0027; A61M 2021/0022; A61M 2021/0016; A61B 5/024
USPC 600/22

(57) ABSTRACT

A womb mimicking neonate's incubator comprising an inner neonate accommodating volume and at least one environment-changing system (ECS). The inner neonate accommodating volume is characterized by at least one parameter and the ECS is configured to change at least one parameter in a predetermined time cycle which mimics human inner-womb environment.

### FIELD OF THE INVENTION

The present invention generally pertains to an incubator that mimics the environment of the womb. More specifically, the invention relates to an incubator that mimics light, sound and movement conditions of the womb either by passively filtering noise and light from the environment or by actively providing sound, light and motion to an incubator isolated from the environment.

### BACKGROUND OF THE INVENTION

An incubator is an apparatus used to accommodate babies born in preterm births to maintain environmental conditions suitable for them. The incubator provides sophisticated measurement means for constantly observing the neonate's temperature, respiration, cardiac function, oxygenation, and brain activity. The incubator also provides means for controlling the temperature, oxygenation, fluid balance and humidity of the neonate and its surrounding as well as to provide him with medications and nutrients.

The incubator is most commonly located in the neonatal intensive care unit (NICU) of a hospital, which its environment is extremely stressful as it is brightly lighted 24 hours a day and there is extensive noise of staff and beeping machines. On top of the environment of the NICU which is stressful even for staff the neonate is also subjected to isolation from human contact and to painful medical procedures.

It is a well-known fact that stress delays and even prevents neonates from getting better. It delays their brain development, gain weight and may increase respiratory and cardiac stress. It has been shown that calm environment with soothing lights and pleasant sounds dramatically improves the physical condition of the neonate. In addition it was proved that skin contact and closeness to a parent (especially the mother) may dramatically improve the medical state of the neonate.

Over the last 10 years or so, NICUs have become much more ‘parent friendly’, encouraging maximum involvement with the babies. Routine gowns and masks are gone and parents are encouraged to help with care as much as possible. Cuddling and skin-to-skin contact, also known as Kangaroo care, are seen as beneficial for all but the frailest. Less stressful ways of delivering high-technology medicine to tiny patients have been devised: sensors to measure blood oxygen levels through the skin, for example; and ways of reducing the amount of blood taken for tests.

A real solution for providing a neonate with a constant safe and calm environment is still missing. Despite the advances of technology that provides less intimidating medical devices and ways to involve parents the neonate is still subjected to an environmental stress.

It is thus still a long felt and unmet need to provide means for keeping the environment of the neonate safe, calm and familiar whilst not compromising on the medical care he is receiving.

### SUMMARY OF THE INVENTION

According to one embodiment of the present invention, a womb mimicking neonate's incubator, wherein said incubator comprises an inner neonate accommodating volume and at least one environment-changing system (ECS); the volume is characterized by at least one parameter; the ECS is configured to change at least one parameter in a predetermined time cycle which mimics human inner-womb environment.

According to an embodiment of the present invention, a neonate's incubator, comprising: (a) an inner volume adapted to accommodate a neonate; the inner volume is characterized by a defined environment; (b) an envelope enclosing the inner volume; and, (c) screening means for filtering at least one parameter of the surrounding environment to create the defined environment; wherein the defined environment mimics the inner-womb environment.

According to an embodiment of the present invention, a womb mimicking neonate's incubator, wherein the incubator comprises an inner neonate accommodating volume, at least one environment-changing system (ECS) and at least one feedback system; the volume is characterized by at least one parameter; the feedback system determines said neonate's stress level; the ECS is adapted to change at least one parameter in response to the feedback system to decrease said levels of stress.

According to another embodiment of the present invention, wherein at least one parameter is selected from a group consisting of spectrum of light, intensity of light, scattering of light, directionality of light, light's daily profile; spectrum of sound, amplitude of sounds, intensity of sound, directionality of sound, type of sound, sound's daily profile, rhythm of motion, intensity of motion, directionality of motion, motion's daily profile, scent, sensation, humidity, temperature and any combination thereof.

According to another embodiment of the present invention, wherein the ECS is selected from a group consisting of passive ECS (PECS), active ECS (AECS), and any combination thereof.

According to another embodiment of the present invention, wherein the AECS is selected from a group consisting of: at least one light system, at least one sound system, at least one scent creator, at least one movement system, bedding and any combination thereof.

According to another embodiment of the present invention, wherein the incubator comprises PECS and AECS.

According to another embodiment of the present invention, wherein the PECS isolates the inner volume from the surrounding environment.

According to another embodiment of the present invention, wherein the PECS blocks from the surrounding environment at least one selected from a group consisting of: motion, light, sound, scent and any combination thereof.

According to another embodiment of the present invention, wherein the PECS blocks about 100% of the surrounding environment's light.

According to another embodiment of the present invention, wherein the PECS is selected from a group consisting of lightproof cover, lightproof room, and any combination thereof.

According to another embodiment of the present invention, wherein the PECS blocks about 100% of the surrounding environment's sound.

According to a preferred embodiment, the inner volume will be completely insulated to sound or insulated to an extent that a normal person cannot detect noise.

According to another embodiment of the present invention, wherein the PECS is selected from soundproof cover, soundproof room, and any combination thereof.

According to another embodiment of the present invention, wherein the PECS blocks about 100% of movement resulting from the surrounding environment.

In a preferred embodiment, the PECS should block movement arising from movement resulting from relocating the incubator or any other operations done on the incubator (opening of its openings, maintenance of the apparatus comprising the incubator, etc.).

According to another embodiment of the present invention, wherein the AECS is selected from a group consisting of: at least one light system, at least one sound system, at least one scent creator, at least one movement system, bedding and any combination thereof.

According to another embodiment of the present invention, wherein the light system comprises at least one light source selected from a group consisting of: incandescent light bulb, halogen lamp, fluorescent lamp, LED lamp, carbon arc lamp, discharge lamp, and any combination thereof.

According to another embodiment of the present invention, wherein the light system comprises at least two light sources.

According to another embodiment of the present invention, wherein at least two light sources differ in at least one selected from a group consisting of amount of light, spectrum of light, directionality of light, type of light source, location, and any combination thereof.

According to another embodiment of the present invention, wherein at least one light source is located in a location selected from a group consisting of upper part of said incubator, lower part of said incubator, side parts of said incubator, outside said incubator and any combination thereof.

According to another embodiment of the present invention, wherein the light system provides light with directionality.

According to another embodiment of the present invention, wherein at least one light source is constantly lit.

According to another embodiment of the present invention, wherein at least one light source provides an amount and spectrum of light equal to that penetrating the womb.

According to another embodiment of the present invention, wherein at least one light source provides light within a specific spectrum.

According to another embodiment of the present invention, wherein at least one light source provides light within the red spectrum.

According to another embodiment of the present invention, wherein at least one light source provides light in a constant amount.

According to another embodiment of the present invention, wherein at least one light source provides light in a changing amount.

According to another embodiment of the present invention, wherein the amount of light provided by at least one light source changes in a cycle.

According to another embodiment of the present invention, wherein the cycle is selected from a group consisting of 24 hours cycle, yearly cycle and any combination thereof.

According to another embodiment of the present invention, wherein the cycle is accommodated to the light exposure cycle of the womb of the woman who carried the neonate.

According to another embodiment of the present invention, wherein the light system is a learning system; the learning system adjusts itself according to the neonate's needs.

According to another embodiment of the present invention, wherein the light system additionally comprises at least one processor and at least one database.

According to another embodiment of the present invention, wherein the database comprising different light cycles.

According to another embodiment of the present invention, wherein at least one sound system comprises at least one speaker located at the inner volume in a location selected from a group consisting of upper part of the incubator, lower part of the incubator, side parts of the incubator, and any combination thereof.

According to another embodiment of the present invention, wherein the sound system provides directionality of sound.

According to another embodiment of the present invention, wherein the sound system plays sounds heard in the womb.

According to another embodiment of the present invention, wherein the sounds heard in the womb are selected from a group consisting of: heartbeat sounds, breathing sounds, gastrointestinal tract sounds, and any combination thereof.

According to another embodiment of the present invention, wherein the sounds heard in the womb are muffled outside noises as heard from within the womb.

According to another embodiment of the present invention, wherein the outside noises are selected from a group consisting of the mother's voice, father's voice, noises of the environment of the mother, and any combination thereof.

According to another embodiment of the present invention, wherein the sound provided by at least one sound system changes in a cycle of about 24 hours.

According to another embodiment of the present invention, wherein the cycle mimics a daily routine of the mother; the sounds changes in a manner selected from a group consisting of: type of noise, intensity of noise, directionality of noise, and any combination thereof.

According to another embodiment of the present invention, wherein the sound system comprises at least two speakers.

According to another embodiment of the present invention, wherein at least two speakers provide different sounds.

According to another embodiment of the present invention, wherein at least two speakers provide different strength of volume.

According to another embodiment of the present invention, wherein the sound system is connected to the mother; the sound system provides live muffled sounds of the mother.

According to another embodiment of the present invention, wherein the sound system comprises a sound database.

According to another embodiment of the present invention, wherein the sound system is a learning system; the learning system adjusts itself according to the neonate's needs.

According to another embodiment of the present invention, wherein the bedding has curves and bumps which mimics the curvatures of the womb.

According to another embodiment of the present invention, wherein the bedding is made of materials mimicking the feeling of the womb's skin.

According to another embodiment of the present invention, wherein the bedding is a water bed; the water bed gives the neonate the feeling of movement within water.

According to another embodiment of the present invention, wherein the bedding is filled with a liquid of changing viscosity; the change in viscosity controls the movement of the bedding and can be accommodated to the neonate's needs.

According to another embodiment of the present invention, wherein the viscosity changes in a manner selected from a group consisting of: manually, as a response to an indication from the neonate, and any combination thereof.

According to another embodiment of the present invention, wherein the indication is selected from a group consisting of: breathing, pulse, blood pressure, sweating and any combination thereof.

According to another embodiment of the present invention, wherein the bedding contains a pocket for providing pseudo-kangaroo care.

According to another embodiment of the present invention, wherein the bedding is made of accommodating materials; the accommodating materials accommodate to at least one selected from a group consisting of: the posture of the neonate, the center of gravity of the neonate, and any combination thereof.

According to another embodiment of the present invention, wherein the movement system moves in at least one manner selected from a group consisting of: rotational movement, vertical movement, horizontal movement, vibrational movement, and any combination thereof.

According to another embodiment of the present invention, wherein movement provided by the movement system can be controlled by at least one manner selected from a group consisting of speed, directionality, type of movement, degree of movement, and any combination thereof.

According to another embodiment of the present invention, wherein the movement provided by the movement system changes in a cycle of about 24 hours.

According to another embodiment of the present invention, wherein the cycle mimics a daily routine of the mother; the movement changes in a manner selected from a group consisting of: type of movement, intensity of movement, and any combination thereof.

According to another embodiment of the present invention, wherein the PECS alters at least one specific parameter in the surrounding environment to mimic the inner-womb environment in the inner volume.

According to another embodiment of the present invention, wherein the PECS comprises at least one filter for screening out from the surrounding environment at least one selected from a group consisting of: motion intensity, light intensity, light spectrum, sound intensity, sound spectrum, and any combination thereof.

According to another embodiment of the present invention, wherein the filter is integrated within at least part of the envelope.

According to another embodiment of the present invention, wherein the filter is adjustable.

According to another embodiment of the present invention, wherein at least one filter reduces light penetration from the surrounding environment.

According to another embodiment of the present invention, wherein at least one filter reduces light penetration to about X % of the surrounding environment's light.

According to another embodiment of the present invention, wherein X equals about 0.1% to about 1%.

According to another embodiment of the present invention, wherein at least one filter provides light in a specific spectrum.

According to another embodiment of the present invention, wherein at least one filter provides light in the red spectrum.

According to another embodiment of the present invention, wherein the PECS reduces sounds from the surrounding environment.

According to another embodiment of the present invention, wherein the PECS is selected from a group consisting of sound filter, sound muffler, sound softener, and any combination thereof.

According to another embodiment of the present invention, wherein the PECS enables penetration of sound in a specific frequency spectrum.

According to another embodiment of the present invention, wherein the PECS enable penetration of low frequency sound into the inner volume.

According to another embodiment of the present invention, wherein the PECS enable penetration of vowel sounds into the inner volume and screens out consonant sounds.

According to another embodiment of the present invention, wherein the PECS comprise a shock absorber; the shock absorber reduces about 100% of movement resulting from the surrounding environment.

According to another embodiment of the present invention, wherein the incubator additionally comprises a feedback system for determining the neonate's level of stress.

According to another embodiment of the present invention, wherein the level of stress is determined according to at least one parameter selected from a group consisting of: pulse, blood pressure, breathing pattern, electrical resistance, sweating and any combination thereof.

According to another embodiment of the present invention, wherein the ECS is activated in response to the feedback system.

According to an embodiment of the present invention, a method for mimicking inner womb environment within a neonate's incubator; the method is characterized by steps of: (a) obtaining an incubator comprising an inner neonate accommodating volume and at least one environment-changing system (ECS); (b) characterizing the volume by at least one parameter; and, (c) changing at least one parameter in a predetermined time cycle, thereby mimicking, within said volume, said inner-womb environment.

According to an embodiment of the present invention, a method for mimicking inner womb environment within a neonate's incubator; said method is characterized by steps of: (a) obtaining an incubator comprising an inner neonate accommodating volume and at least one environment-changing system (ECS) and at least one feedback system; (b) characterizing said volume by at least one parameter; (c) determining said neonate's stress level by said feedback system; and, (d) changing said at least one parameter in response to said feedback system, thereby decreasing said levels of neonate's stress.

According to an embodiment of the present invention, a method for changing a defined environment within a neonate's incubator, comprising steps of: (a) obtaining a neonate's incubator comprising an inner volume and an envelope enclosing the inner volume; the inner volume is adapted to accommodate a neonate; (b) inserting the incubator into isolation means; the isolation means isolates the inner volume from the environment of the surrounding environment; (c) embedding or otherwise connecting at least one environment changing system (ECS) to the incubator; and, (d) changing at least one parameter of the defined environment by the ECS; wherein the defined environment is mimicking the inner-womb environment.

According to an embodiment of the present invention, a method for changing a defined environment within a neonate's incubator, comprising steps of: (a) obtaining a neonate's incubator comprising an inner volume and an envelope enclosing the inner volume; the inner volume is adapted to accommodate a neonate; (b) embedding or otherwise connecting screening means to the incubator for filtering parameters of the surrounding environment to create the defined environment; and, (c) screening at least one parameter of the defined environment by the screening means; wherein the defined environment is mimicking the inner-womb environment.

According to another embodiment of the present invention, additionally comprising a step of selecting at least one parameter from a group consisting of spectrum of light, intensity of light, scattering of light, directionality of light, light's daily profile; spectrum of sound, amplitude of sounds, intensity of sound, directionality of sound, type of sound, sound's daily profile, rhythm of motion, intensity of motion, directionality of motion, motion's daily profile, scent, sensation, humidity, temperature and any combination thereof.

According to another embodiment of the present invention, additionally comprising a step of selecting the ECS from a group consisting of passive ECS (PECS), active ECS (AECS), and any combination thereof.

According to another embodiment of the present invention, additionally comprising a step of selecting the AECS from a group consisting of: at least one light system, at least one sound system, at least one scent creator, at least one movement system, bedding and any combination thereof.

According to another embodiment of the present invention, wherein the incubator comprises a PECS and an AECS.

According to another embodiment of the present invention, additionally comprising a step of isolating the inner volume from the surrounding environment by the PECS.

According to another embodiment of the present invention, additionally comprising a step of blocking by the PECS from the surrounding environment at least one selected from a group consisting of: motion, light, sound, scent and any combination thereof.

According to another embodiment of the present invention, additionally comprising a step of blocking about 100% of the surrounding environment's light by the PECS.

According to another embodiment of the present invention, additionally comprising a step of selecting the PECS from a group consisting of lightproof cover, lightproof room, and any combination thereof.

According to another embodiment of the present invention, additionally comprising a step of blocking about 100% of the surrounding environment's sound by blocking the PECS.

According to another embodiment of the present invention, additionally comprising a step of selecting the PECS from soundproof cover, soundproof room, and any combination thereof.

According to another embodiment of the present invention, additionally comprising a step of blocking about 100% of movement resulting from the surrounding environment.

According to another embodiment of the present invention, additionally comprising a step of selecting the AECS from a group consisting of: at least one light system, at least one sound system, at least one scent creator, at least one movement system, bedding and any combination thereof.

According to another embodiment of the present invention, wherein the light system comprises at least one light source selected from a group consisting of: incandescent light bulb, halogen lamp, fluorescent lamp, LED lamp, carbon arc lamp, discharge lamp, and any combination thereof.

According to another embodiment of the present invention, wherein the light system comprises at least two light sources.

According to another embodiment of the present invention, wherein at least two light sources differ in at least one selected from a group consisting of amount of light, spectrum of light, directionality of light, type of light source, location, and any combination thereof.

According to another embodiment of the present invention, additionally comprising a step of locating at least one light source at the inner volume in a location selected from a group consisting of upper part of the incubator, lower part of the incubator, side parts of the incubator, and any combination thereof.

According to another embodiment of the present invention, additionally comprising a step of providing light with directionality by the light system provides.

According to another embodiment of the present invention, additionally comprising a step of constantly lighting at least one light source.

According to another embodiment of the present invention, additionally comprising a step of providing an amount and spectrum of light equal to that penetrating the womb by at least one light source provides.

According to another embodiment of the present invention, additionally comprising a step of providing light within a specific spectrum.

According to another embodiment of the present invention, additionally comprising a step of providing light within the red spectrum.

According to another embodiment of the present invention, additionally comprising a step of providing light in a constant amount.

According to another embodiment of the present invention, additionally comprising a step of providing light in a changing amount.

According to another embodiment of the present invention, additionally comprising a step of providing changing amounts of light in a cycle.

According to another embodiment of the present invention, additionally comprising a step of selecting the cycle from a group consisting of 24 hours cycle, yearly cycle and any combination thereof.

According to another embodiment of the present invention, additionally comprising a step of accommodating the cycle to the light exposure cycle of the womb of the woman who carried the neonate.

According to another embodiment of the present invention, wherein the light system is a learning system; the learning system is adjusting itself according to the neonate's needs.

According to another embodiment of the present invention, wherein the light system additionally comprises at least one processor and at least one database.

According to another embodiment of the present invention, wherein the database comprising different light cycles.

According to another embodiment of the present invention, wherein at least one sound system comprises at least one speaker located at the inner volume in a location selected from a group consisting of upper part of the incubator, lower part of the incubator, side parts of the incubator, and any combination thereof.

According to another embodiment of the present invention, wherein the sound system provides directionality of sound.

According to another embodiment of the present invention, additionally comprising a step of playing sounds heard in the womb.

According to another embodiment of the present invention, additionally comprising a step of selecting the sounds heard in the womb from a group consisting of: heartbeat sounds, breathing sounds, gastrointestinal tract sounds, and any combination thereof.

According to another embodiment of the present invention, additionally comprising a step of muffling outside noises as heard from within the womb.

According to another embodiment of the present invention, additionally comprising a step of selecting the outside noises from a group consisting of the mother's voice, father's voice, noises of the environment of the mother, and any combination thereof.

According to another embodiment of the present invention, additionally comprising a step of changing the sound provided in a cycle of about 24 hours.

According to another embodiment of the present invention, additionally comprising a step of mimicking a daily routine of the mother; the sounds changes in a manner selected from a group consisting of: type of noise, intensity of noise, directionality of noise, and any combination thereof.

According to another embodiment of the present invention, wherein the sound system comprises at least two speakers.

According to another embodiment of the present invention, additionally comprising a step of providing different sounds by at least two speakers provide.

According to another embodiment of the present invention, additionally comprising a step of providing different strength of volume by at least two speakers provide.

According to another embodiment of the present invention, additionally comprising a step of connecting the sound system to the mother and providing live muffled sounds of the mother.

According to another embodiment of the present invention, wherein the sound system comprises a sound database.

According to another embodiment of the present invention, wherein the sound system is a learning system; the learning system is adjusting itself according to the neonate's needs.

According to another embodiment of the present invention, additionally comprising a step of adding to the bedding curves and bumps which mimics the curvatures of the womb.

According to another embodiment of the present invention, additionally comprising a step of making the bedding of materials mimicking the feeling of the womb's skin.

According to another embodiment of the present invention, wherein the bedding is a water bed; the water bed is giving the neonate the feeling of movement within water.

According to another embodiment of the present invention, additionally comprising a step of filling the bedding with a liquid of changing viscosity; the change in viscosity controls the movement of the bedding and can be accommodated to the neonate's needs.

According to another embodiment of the present invention, additionally comprising a step of changing the viscosity in a manner selected from a group consisting of: manually, as a response to an indication from the neonate, and any combination thereof.

According to another embodiment of the present invention, additionally comprising a step of selecting the indication from a group consisting of: breathing, pulse, blood pressure, sweating and any combination thereof.

According to another embodiment of the present invention, additionally comprising a step of adding to the bedding a pocket for providing pseudo-kangaroo care.

According to another embodiment of the present invention, additionally comprising a step of making the bedding from accommodating materials; the accommodating materials accommodate to at least one selected from a group consisting of: the posture of the neonate, the center of gravity of the neonate, and any combination thereof.

According to another embodiment of the present invention, wherein additionally comprising a step of moving the incubator by the movement system in at least one manner selected from a group consisting of: rotational movement, vertical movement, horizontal movement, vibrational movement, and any combination thereof.

According to another embodiment of the present invention, additionally comprising a step of controlling the movement provided by the movement system by at least one manner selected from a group consisting of speed, directionality, type of movement, degree of movement, and any combination thereof.

According to another embodiment of the present invention, additionally comprising a step of changing the movement provided by the movement system changes in a cycle of about 24 hours.

According to another embodiment of the present invention, additionally comprising a step of mimicking a daily routine of the mother; the movement changes in a manner selected from a group consisting of: type of movement, intensity of movement, and any combination thereof.

According to another embodiment of the present invention, additionally comprising a step of altering by the PECS at least one specific parameter in the surrounding environment to mimic the inner-womb environment in the inner volume.

According to another embodiment of the present invention, wherein the PECS comprises at least one filter for screening out from the surrounding environment at least one selected from a group consisting of: motion intensity, light intensity, light spectrum, sound intensity, sound spectrum, and any combination thereof.

According to another embodiment of the present invention, additionally comprising a step of integrating the filter within at least part of the envelope.

According to another embodiment of the present invention, additionally comprising a step of adjusting the filter.

According to another embodiment of the present invention, additionally comprising a step of reducing by at least one filter light penetration from the surrounding environment.

According to another embodiment of the present invention, additionally comprising a step of reducing by at least one filter light penetration to about X % of the surrounding environment's light.

According to another embodiment of the present invention, wherein X equals about 0.1% to about 1%.

According to another embodiment of the present invention, additionally comprising a step of providing light in a specific spectrum.

The method of 63, additionally comprising a step of providing light in the red spectrum.

According to another embodiment of the present invention, additionally comprising a step of reducing sounds from the surrounding environment.

According to another embodiment of the present invention, additionally comprising a step of selecting the PECS from a group consisting of sound filter, sound muffler, sound softener, and any combination thereof.

According to another embodiment of the present invention, additionally comprising a step of enabling penetration of sound in a specific frequency spectrum.

According to another embodiment of the present invention, additionally comprising a step of enabling penetration of low frequency sound into the inner volume.

According to another embodiment of the present invention, additionally comprising a step of enabling penetration of vowel sounds into the inner volume and screens out consonant sounds.

According to another embodiment of the present invention, wherein the PECS comprise a shock absorber; the shock absorber is reducing about 100% of movement resulting from the surrounding environment.

According to another embodiment of the present invention, additionally comprising a step of attaching to the incubator a feedback system for determining the neonate's level of stress.

According to another embodiment of the present invention, additionally comprising a step of determining the level of stress according to at least one parameter selected from a group consisting of: pulse, blood pressure, breathing pattern, electrical resistance, sweating and any combination thereof.

According to another embodiment of the present invention, additionally comprising a step of activating the ECS in response to the feedback system.

### BRIEF DESCRIPTION OF THE FIGURES

In the following detailed description of the preferred embodiments, reference is made to the accompanying drawings that form a part hereof, and in which are shown by way of illustration specific embodiments in which the invention may be practiced. It is understood that other embodiments may be utilized and structural changes may be made without departing from the scope of the present invention. The present invention may be practiced according to the claims without some or all of these specific details. For the purpose of clarity, technical material that is known in the technical fields related to the invention has not been described in detail so that the present invention is not necessarily obscured. In the accompanying drawing:

FIG. 1 is an illustration of a preferred embodiment of a neonate's incubator having various AECS such as a sound system, a light system and a movement system;

FIGS. 2A and 2B are illustrations of preferred embodiments of a neonate's incubator;

FIG. 2A illustrates the neonate's incubator cover having no filters for light;

FIG. 2B illustrates the neonate's incubator having a cover that filters light from the outside environment;

FIGS. 3A and 3B are illustrations of preferred embodiments of a neonate's incubator having different movement systems;

FIG. 4 is a schematic flow diagram describing a preferred method for mimicking inner womb environment within a neonate's incubator;

FIG. 5 is a schematic flow diagram describing another preferred method for changing a defined environment within a neonate's incubator to mimic the inner-womb environment;

FIG. 6 is a schematic flow diagram describing another preferred method for changing a defined environment within a neonate's incubator to mimic the inner-womb environment; and,

FIG. 7 is a schematic flow diagram describing a preferred method for mimicking inner womb environment within a neonate's incubator.

### DETAILED DESCRIPTION OF THE PREFERRED EMBODIMENTS

The following description is provided, alongside all chapters of the present invention, so as to enable any person skilled in the art to make use of the invention and set forth the best modes contemplated by the inventor of carrying out this invention. Various modifications, however, will remain apparent to those skilled in the art, since the generic principles of the present invention is defined to specifically provide a neonate's incubator having an inner environment mimicking the inner-womb environment for providing the neonate the best conditions for development and strengthening. The womb like environment is provided by screening means that screens the outer environment (mainly light, sound and movement) and accommodates it to be as close as possible to the environment of the womb. It is also possible to completely isolate the incubator from the outer environment and create an womb mimicking environment by artificial means such as light, sound and movement systems.

According to one embodiment of the present invention, a womb mimicking neonate's incubator, wherein said incubator comprises an inner neonate accommodating volume and at least one environment-changing system (ECS); the volume is characterized by at least one parameter; the ECS is configured to change at least one parameter in a predetermined time cycle which mimics human inner-womb environment.

According to an embodiment of the present invention, a neonate's incubator, comprising: (a) an inner volume adapted to accommodate a neonate; the inner volume is characterized by a defined environment; (b) an envelope enclosing the inner volume; and, (c) screening means for filtering at least one parameter of the surrounding environment to create the defined environment; wherein the defined environment mimics the inner-womb environment.

According to an embodiment of the present invention, a womb mimicking neonate's incubator, wherein the incubator comprises an inner neonate accommodating volume, at least one environment-changing system (ECS) and at least one feedback system; the volume is characterized by at least one parameter; the feedback system determines said neonate's stress level; the ECS is adapted to change at least one parameter in response to the feedback system to decrease said levels of stress.

According to another embodiment of the present invention, wherein at least one parameter is selected from a group consisting of spectrum of light, intensity of light, scattering of light, directionality of light, light's daily profile; spectrum of sound, amplitude of sounds, intensity of sound, directionality of sound, type of sound, sound's daily profile, rhythm of motion, intensity of motion, directionality of motion, motion's daily profile, scent, sensation, humidity, temperature and any combination thereof.

In a preferred embodiment, The ECS changes a parameter or parameters of the outer environment and enables its penetration to the inner volume of the incubator in a way the mimics the inner womb environment.

The ECS may be connected to sensors which transmits information regarding the stress level of the neonate. The system may change her operations as a reaction to the stress level. The system may be a self-learning system that learns how to react to stress of specific neonates. In another embodiment, it may be programmed to start different patterns according to different levels of stress.

In another preferred embodiment, the ECS itself creates the parameter to create an environment that mimics the inner-womb environment. In this embodiment, the incubator has to be isolated from the outer environment at least with regards to the parameter which is created. For example, it will be futile to create a light in a specific spectrum and intensity while the incubator is situated in an intensive care unit lit with strong and bright light. Therefore, the solution is to block the incubator from all surrounding lights.

According to another embodiment of the present invention, wherein the ECS is selected from a group consisting of passive ECS (PECS), active ECS (AECS), and any combination thereof.

A PECS relates to an ECS that changes a parameter or parameters of the outer environment. For example, a light filter that introduces only a specific spectrum of light from the outer environment, a sound muffler that muffle outside sounds, a shock absorber that reduces the movement of the incubator while it is in transit. A PECS may additionally block the parameter of the outer environment in the purpose of enabling the usage of a AECS.

An AECS relates to an ECS that creates the parameter itself. For example a light system, an olfactory system, a sound system, a movement system and bedding. In most cases, the incubator has to be isolated from the outer environment in order for the AECS to be effective.

According to another embodiment of the present invention, wherein the AECS is selected from a group consisting of: at least one light system, at least one sound system, at least one scent creator, at least one movement system, bedding and any combination thereof.

The AECS may also include an olfactory system that creates an olfactory perception similar to that on the womb.

According to another embodiment of the present invention, wherein the incubator comprises a PECS and an AECS.

In a preferred embodiment in which the incubator comprises both a PECS and an AECS, the role of the PECS is to block a parameter from the outer environment which is created by the AECS. For example, if the parameter is a spectrum of light—than the PECS prevents penetration of light from the outer environment and the AECS produces light in a specific spectrum.

According to another embodiment of the present invention, wherein the PECS isolates the inner volume from the surrounding environment.

The PECS may isolate the inner volume from the outer environment regarding one parameter or few parameters. For example, it can prevent penetration of light or sound or light and sound.

According to another embodiment of the present invention, wherein the PECS blocks from the surrounding environment at least one selected from a group consisting of: motion, light, sound, scent and any combination thereof.

According to another embodiment of the present invention, wherein the PECS blocks about 100% of the surrounding environment's light.

This means that no light enters the inner volume from the outer environment. If there is no light system the inner volume will be in complete dark with no relation to the amount of light in the outside environment.

According to another embodiment of the present invention, wherein the PECS is selected from a group consisting of lightproof cover, lightproof room, and any combination thereof.

In a preferred embodiment, the lightproof cover covers the whole incubator. It can be in the form of a box in which the incubator is located.

In another preferred embodiment, the lightproof cover covers only the parts of the incubator which are transparent to light. It can be a shield hovering on top of the incubator. It can also be movable manually or automatically.

In a preferred embodiment, a lightproof room is a room with no windows and an entrance with a double door or a turning door that enables entering without introducing light within the room and incubator. The room may have lighting in a specific spectrum to enable functioning in it. The specific spectrum has to be in agreement with the specific spectrum in the inner volume.

In another preferred embodiment, the envelope of the incubator which distinguishes the inner volume from the outer environment may of the

According to another embodiment of the present invention, wherein the PECS blocks about 100% of the surrounding environment's sound.

In a preferred embodiment, all the incubator's walls may have the characteristics described or most of the walls may be impermeable to sounds while only an opening or few openings are covered with special materials that screens and/or softens and/or reduces and/or muffles the environment's sounds.

In another embodiment of the invention, the interior of the incubator is impermeable to sounds coming from the outside environment. The incubator walls may be made from soundproof materials and/or the incubator may be covered with sound proof materials and/or the incubator may be placed in a sound proof room.

According to a preferred embodiment, the inner volume will be completely insulated to sound or insulated to an extent that a normal person cannot detect noise.

According to another embodiment of the present invention, wherein the PECS is selected from soundproof cover, soundproof room, and any combination thereof.

The soundproof cover may be a part of the incubator walls or an additional cover to be put over the incubator's walls.

According to another embodiment of the present invention, wherein the PECS blocks about 100% of movement resulting from the surrounding environment.

In a preferred embodiment, the PECS should block movement arising from movement resulting from relocating the incubator or any other operations done on the incubator (opening of its openings, maintenance of the apparatus comprising the incubator, etc.).

According to another embodiment of the present invention, wherein the AECS is selected from a group consisting of: at least one light system, at least one sound system, at least one scent creator, at least one movement system, bedding and any combination thereof.

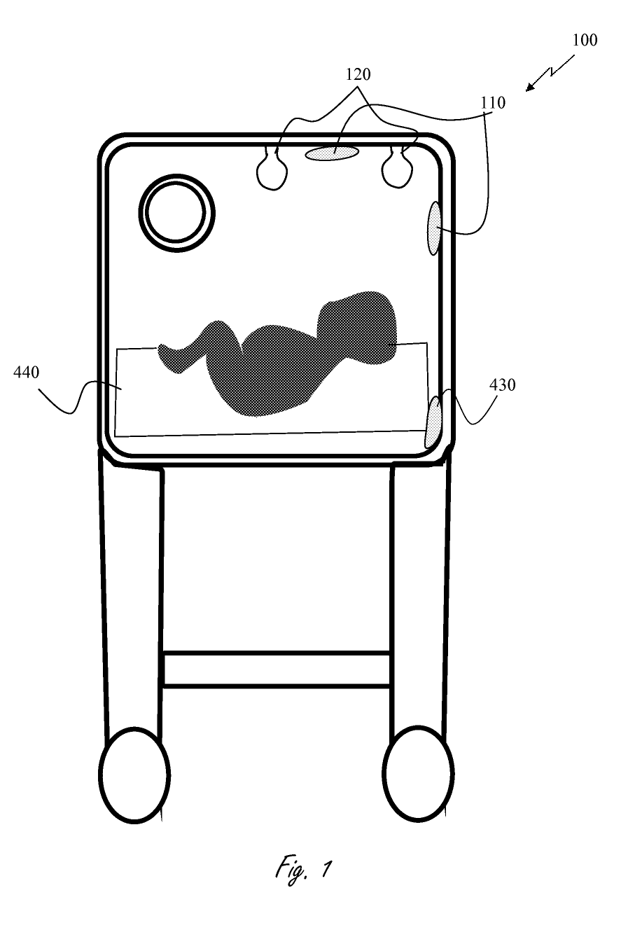

Reference is now made to FIG. 1, which is an illustration of one preferred embodiment of a neonate's incubator (100) having various AECs such as a sound system (110), a light system (120), a movement system (430) and bedding (440).

In a preferred embodiment, after blocking a specific parameter within the inner volume of the incubator from the outer environment, the parameter is artificially introduced. The parameter may be light, sound, movement, scent or any other parameter that may affect the perception of a neonate and make the environment as close as possible to the inner-womb environment.

According to another embodiment of the present invention, wherein the light system comprises at least one light source selected from a group consisting of: incandescent light bulb, halogen lamp, fluorescent lamp, LED lamp, carbon arc lamp, discharge lamp, and any combination thereof.

The light source may provide light in a specific spectrum, direction, intensity, etc.

Reference is still made to FIG. 1, which illustrates an incubator (100) comprising two light sources (120) located within the incubator in the middle and right side of its upper part. However, the light system can be located in other location of the incubator (side, top or bottom) or outside the incubator. More than one light source enables to create directionality of light as well as different light environment.

According to another embodiment of the present invention, wherein the light system comprises at least two light sources. More than two light sources are also a preferable option.

In a preferred embodiment, more than one source can provide a more diverse environment as it can produce, for example, light in to spectrums, each having a different directionality.
It is a well-known fact that a fetus reacts to outside light. Proves for responsiveness to light within the womb can be found in: 1) The relationship between the response to external light stimulation and behavioral states in the human fetus: how it differs from vibroacoustic stimulation. Kiuchi M, Nagata N, Ikeno S, Terakawa N. Early Hum Dev. 2000 May; 58(2):153-65 (currently available at http://www.earlyhumandevelopment.com/article/S0378-3782(00)00074-8/pdf) relevant when fetus is awake; and (2) Direct in utero perception of light by the mammalian fetus. Weaver D R, Reppert S M. Brain Res Dev Brain Res. 1989 May 1; 47(1): 151-5 (currently available at http://www.ncbi.nlm.nih.gov/pubmed/2736762).

According to another embodiment of the present invention, wherein at least two light sources differ in at least one selected from a group consisting of amount of light, spectrum of light, directionality of light, type of light source, location, and any combination thereof.

According to another embodiment of the present invention, wherein at least one light source is located in a location selected from a group consisting of upper part of said incubator, lower part of said incubator, side parts of said incubator, outside said incubator and any combination thereof. Light sources located outside the incubator is preferable as it prevents heating but it should only be introduced with a PECS that does not block the light.

According to another embodiment of the present invention, wherein the light system provides light with directionality. The directionality may mimic the light directionality in the womb as more light penetrates the womb from one side (front abdomen side) than the other side (back side). The directionality also depends on the location of the baby in comparison to its location in the womb. For example, if the face was facing the abdomen within the womb than it received more light than if it was facing the rear.

According to another embodiment of the present invention, wherein at least one light source is constantly lit.

According to another embodiment of the present invention, wherein at least one light source provides an amount and spectrum of light equal to that penetrating the womb.

According to another embodiment of the present invention, wherein at least one light source provides light within a specific spectrum. It has been shown in the paper PENETRATION OF LIGHT INTO THE UTERUS OF PREGNANT MAMMALS Steven L. Jacques1, David R. Weaver, Steven M. Reppert, 2 JAN 2008 Photochemistry and Photobiology Volume 45, Issue 5, pages 637-641, May 1987 that mostly light within the red spectrum penetrates the womb. Therefore, providing light within the red spectrum will mimic the inner womb light environment.

According to another embodiment of the present invention, wherein at least one light source provides light within the red spectrum.

According to another embodiment of the present invention, wherein at least one light source provides light in a constant amount.

According to another embodiment of the present invention, wherein at least one light source provides light in a changing amount.

According to another embodiment of the present invention, wherein the amount of light provided by at least one light source changes in a cycle. The cycle of course has to be imitating as much as possible the cycle of the mother. It should take into considerations the amount of light she was exposed to and in what times. For example, if she wore heavy clothing that blocked light penetration than the fetus was hardly exposed to light even if his mom spent most of her time in bright light.

According to another embodiment of the present invention, wherein the cycle is selected from a group consisting of 24 hours cycle, yearly cycle and any combination thereof. The light cycle the fetus was used to in the womb also depend on seasons of the year (especially in northern and southern countries with big differences between seasons). In a preferred embodiment the cycle comprises a 24 cycle and a yearly cycle. The yearly cycle is less applicable as chances are that the neonate will night stay within the incubator for that long.

According to another embodiment of the present invention, wherein the cycle is accommodated to the light exposure cycle of the womb of the woman who carried the neonate.

According to another embodiment of the present invention, wherein the light system is a learning system; the learning system adjusts itself according to the neonate's needs. The AECS can accommodate itself to the neonate's need. It can be attached to sensors that measure the stress level of the neonate) and change in the purpose to reduce the stress level as much as possible. It can identify patterns the neonate favors or react to crying sounds or increased blood pressure.

According to another embodiment of the present invention, wherein the light system additionally comprises at least one processor and at least one database. The database may include different light patterns and also knowledge about when they are preferred. The patterns may include directionality, spectrum and intensity. In another preferred embodiment new patterns can be introduced to the database.

According to another embodiment of the present invention, wherein the database comprising different light cycles.

According to another embodiment of the present invention, wherein at least one sound system comprises at least one speaker located at the inner volume in a location selected from a group consisting of upper part of the incubator, lower part of the incubator, side parts of the incubator, and any combination thereof.

According to another embodiment of the present invention, wherein the sound system provides directionality of sound. Since there is directionality in sound within the womb it is important to mimic the directionality. Similar to light the sound penetrates mainly through the front side of the womb.

According to another embodiment of the present invention, wherein the sound system plays sounds heard in the womb.

According to another embodiment of the present invention, wherein the sounds heard in the womb are selected from a group consisting of: heartbeat sounds, breathing sounds, gastrointestinal tract sounds, and any combination thereof.

According to another embodiment of the present invention, wherein the sounds heard in the womb are muffled outside noises as heard from within the womb.

According to another embodiment of the present invention, wherein the outside noises are selected from a group consisting of the mother's voice, father's voice, noises of the environment of the mother, and any combination thereof.

According to another embodiment of the present invention, wherein the sound provided by at least one sound system changes in a cycle of about 24 hours.

According to another embodiment of the present invention, wherein the cycle mimics a daily routine of the mother; the sounds changes in a manner selected from a group consisting of: type of noise, intensity of noise, directionality of noise, and any combination thereof.

According to another embodiment of the present invention, wherein the sound system comprises at least two speakers.

According to another embodiment of the present invention, wherein at least two speakers provide different sounds.

According to another embodiment of the present invention, wherein at least two speakers provide different strength of volume.

According to another embodiment of the present invention, wherein the sound system is connected to the mother; the sound system provides live muffled sounds of the mother.

According to another embodiment of the present invention, wherein the sound system comprises a sound database. The database may include muffled outer noises of different environments and can be selected according to the environment the mother was exposed to while carrying the neonate. For example, if she worked in a factory there may be machine sounds.

According to another embodiment of the present invention, wherein the sound system is a learning system; the learning system adjusts itself according to the neonate's needs.

According to another embodiment of the present invention, wherein the bedding has curves and bumps which mimics the curvatures of the womb.

According to another embodiment of the present invention, wherein the bedding is made of materials mimicking the feeling of the womb's skin. The materials may give a pseudo wet feeling.

According to another embodiment of the present invention, wherein the bedding is a water bed; the water bed gives the neonate the feeling of movement within water. The fetus was used to swim within the womb. A water bedding may give him degrees of freedom as he used to have in the womb.

According to another embodiment of the present invention, wherein the bedding is filled with a liquid of changing viscosity; the change in viscosity controls the movement of the bedding and can be accommodated to the neonate's needs. The viscosity can be accommodated to a specific neonate according to his needs and preferences.

According to another embodiment of the present invention, wherein the viscosity changes in a manner selected from a group consisting of: manually, as a response to an indication from the neonate, and any combination thereof. The viscosity may change by a learning system that receives input from sensors sensing stress of the neonate. The viscosity changes until the lowest amount of stress is received. The adjustment of the viscosity can also be changed according manually in a trial and error manner.

According to another embodiment of the present invention, wherein the indication is selected from a group consisting of: breathing, pulse, blood pressure, sweating and any combination thereof.

According to another embodiment of the present invention, wherein the bedding contains a pocket for providing pseudo-kangaroo care. Putting the neonate within a pocket gives him a safe sensation.

According to another embodiment of the present invention, wherein the bedding is made of accommodating materials; the accommodating materials accommodate to at least one selected from a group consisting of: the posture of the neonate, the center of gravity of the neonate, and any combination thereof. Accommodating materials wrap the neonate and mimics sensation of touch.

According to another embodiment of the present invention, wherein the movement system moves in at least one manner selected from a group consisting of: rotational movement, vertical movement, horizontal movement, vibrational movement, and any combination thereof.

According to another embodiment of the present invention, wherein movement provided by the movement system can be controlled by at least one manner selected from a group consisting of speed, directionality, type of movement, degree of movement, and any combination thereof.

According to another embodiment of the present invention, wherein the movement provided by the movement system changes in a cycle of about 24 hours. The movement may mimic the movement habit of the neonate within the womb. The movement within the womb depends on the mother's lifestyle. The movement of the incubator may be accommodated according to the mother's lifestyle.

According to another embodiment of the present invention, wherein the cycle mimics a daily routine of the mother; the movement changes in a manner selected from a group consisting of: type of movement, intensity of movement, and any combination thereof.

According to another embodiment of the present invention, wherein the PECS alters at least one specific parameter in the surrounding environment to mimic the inner-womb environment in the inner volume.

According to another embodiment of the present invention, wherein the PECS comprises at least one filter for screening out from the surrounding environment at least one selected from a group consisting of: motion intensity, light intensity, light spectrum, sound intensity, sound spectrum, and any combination thereof.

According to another embodiment of the present invention, wherein the filter is integrated within at least part of the envelope.

According to another embodiment of the present invention, wherein the filter is adjustable.

According to another embodiment of the present invention, wherein at least one filter reduces light penetration from the surrounding environment.

According to another embodiment of the present invention, wherein at least one filter reduces light penetration to about X % of the surrounding environment's light.

According to another embodiment of the present invention, wherein X equals about 0.1% to about 1%. It was shown that the outside lighting is reduced to provide 0.1%-1% of external luminence in the paper: Del Giudice, M. (2011), Alone in the dark? Modeling the conditions for visual experience in human fetuses. Dev. Psychobiol., 53: 214-219, which is incorporated here as a reference, that this is the amount of light that is transferred to the fetus.

According to another embodiment of the present invention, wherein at least one filter provides light in a specific spectrum.

According to another embodiment of the present invention, wherein at least one filter provides light in the red spectrum.

According to another embodiment of the present invention, wherein the PECS reduces sounds from the surrounding environment.

According to another embodiment of the present invention, wherein the PECS is selected from a group consisting of sound filter, sound muffler, sound softener, and any combination thereof.

According to another embodiment of the present invention, wherein the PECS enables penetration of sound in a specific frequency spectrum.

According to another embodiment of the present invention, wherein the PECS enable penetration of low frequency sound into the inner volume. It has been shown that the sounds entering the incubator from the outside environment are low frequency sounds which penetrates better into the womb. It was shown in a paper currently available at http://www.nature.com/news/2003/030202/full/news040126-17.html, brought here as reference that low frequency sounds penetrates the womb better than high frequency sounds. This paper also proves that vowels sounds penetrates better than constants sounds and therefore screening may also be made for vowel sounds.

According to another embodiment of the present invention, wherein the PECS enable penetration of vowel sounds into the inner volume and screens out consonant sounds.

According to another embodiment of the present invention, wherein the PECS comprise a shock absorber; the shock absorber reduces about 100% of movement resulting from the surrounding environment.

According to another embodiment of the present invention, wherein the incubator additionally comprises a feedback system for determining the neonate's level of stress.

According to another embodiment of the present invention, wherein the level of stress is determined according to at least one parameter selected from a group consisting of: pulse, blood pressure, breathing pattern, electrical resistance, sweating and any combination thereof.

According to another embodiment of the present invention, wherein the ECS is activated in response to the feedback system.

According to an embodiment of the present invention, a method for mimicking inner womb environment within a neonate's incubator; said method is characterized by steps of: (a) obtaining an incubator comprising an inner neonate accommodating volume and at least one environment-changing system (ECS) and at least one feedback system; (b) characterizing said volume by at least one parameter; (c) determining said neonate's stress level by said feedback system; and, (d) changing said at least one parameter in response to said feedback system, thereby decreasing said levels of neonate's stress.

According to an embodiment of the present invention, a method for changing a defined environment within a neonate's incubator, comprising steps of: (a) obtaining a neonate's incubator comprising an inner volume and an envelope enclosing the inner volume; the inner volume is adapted to accommodate a neonate; (b) inserting the incubator into isolation means; the isolation means isolates the inner volume from the environment of the surrounding environment; (c) embedding or otherwise connecting at least one environment changing system (ECS) to the incubator; and, (d) changing at least one parameter of the defined environment by the ECS; wherein the defined environment is mimicking the inner-womb environment.

According to an embodiment of the present invention, a method for changing a defined environment within a neonate's incubator, comprising steps of: (a) obtaining a neonate's incubator comprising an inner volume and an envelope enclosing the inner volume; the inner volume is adapted to accommodate a neonate; (b) embedding or otherwise connecting screening means to the incubator for filtering parameters of the surrounding environment to create the defined environment; and, (c) screening at least one parameter of the defined environment by the screening means; wherein the defined environment is mimicking the inner-womb environment.

According to another embodiment of the present invention, additionally comprising a step of selecting at least one parameter from a group consisting of spectrum of light, intensity of light, scattering of light, directionality of light, light's daily profile; spectrum of sound, amplitude of sounds, intensity of sound, directionality of sound, type of sound, sound's daily profile, rhythm of motion, intensity of motion, directionality of motion, motion's daily profile, scent, sensation, humidity, temperature and any combination thereof. According to another embodiment of the present invention, additionally comprising a step of selecting the ECS from a group consisting of passive ECS (PECS), active ECS (AECS), and any combination thereof.

According to another embodiment of the present invention, additionally comprising a step of selecting the AECS from a group consisting of: at least one light system, at least one sound system, at least one scent creator, at least one movement system, bedding and any combination thereof.

According to another embodiment of the present invention, wherein the incubator comprises a PECS and an AECS.

According to another embodiment of the present invention, additionally comprising a step of isolating the inner volume from the surrounding environment by the PECS.

According to another embodiment of the present invention, additionally comprising a step of blocking by the PECS from the surrounding environment at least one selected from a group consisting of: motion, light, sound, scent and any combination thereof.

According to another embodiment of the present invention, additionally comprising a step of blocking about 100% of the surrounding environment's light by the PECS.

According to another embodiment of the present invention, additionally comprising a step of selecting the PECS from a group consisting of lightproof cover, lightproof room, and any combination thereof.

According to another embodiment of the present invention, additionally comprising a step of blocking about 100% of the surrounding environment's sound by blocking the PECS.

According to another embodiment of the present invention, additionally comprising a step of selecting the PECS from soundproof cover, soundproof room, and any combination thereof.

According to another embodiment of the present invention, additionally comprising a step of blocking about 100% of movement resulting from the surrounding environment.

According to another embodiment of the present invention, additionally comprising a step of selecting the AECS from a group consisting of: at least one light system, at least one sound system, at least one scent creator, at least one movement system, bedding and any combination thereof.

According to another embodiment of the present invention, wherein the light system comprises at least one light source selected from a group consisting of: incandescent light bulb, halogen lamp, fluorescent lamp, LED lamp, carbon arc lamp, discharge lamp, and any combination thereof.

According to another embodiment of the present invention, wherein the light system comprises at least two light sources.

According to another embodiment of the present invention, wherein at least two light sources differ in at least one selected from a group consisting of amount of light, spectrum of light, directionality of light, type of light source, location, and any combination thereof.

According to another embodiment of the present invention, additionally comprising a step of locating at least one light source at the inner volume in a location selected from a group consisting of upper part of the incubator, lower part of the incubator, side parts of the incubator, and any combination thereof.

According to another embodiment of the present invention, additionally comprising a step of providing light with directionality by the light system provides.

According to another embodiment of the present invention, additionally comprising a step of constantly lighting at least one light source.

According to another embodiment of the present invention, additionally comprising a step of providing an amount and spectrum of light equal to that penetrating the womb by at least one light source provides.

According to another embodiment of the present invention, additionally comprising a step of providing light within a specific spectrum.

According to another embodiment of the present invention, additionally comprising a step of providing light within the red spectrum.

According to another embodiment of the present invention, additionally comprising a step of providing light in a constant amount.

According to another embodiment of the present invention, additionally comprising a step of providing light in a changing amount.

According to another embodiment of the present invention, additionally comprising a step of providing changing amounts of light in a cycle.

According to another embodiment of the present invention, additionally comprising a step of selecting the cycle from a group consisting of 24 hours cycle, yearly cycle and any combination thereof.

According to another embodiment of the present invention, additionally comprising a step of accommodating the cycle to the light exposure cycle of the womb of the woman who carried the neonate.

According to another embodiment of the present invention, wherein the light system is a learning system; the learning system is adjusting itself according to the neonate's needs.

According to another embodiment of the present invention, wherein the light system additionally comprises at least one processor and at least one database.

According to another embodiment of the present invention, wherein the database comprising different light cycles.

According to another embodiment of the present invention, wherein at least one sound system comprises at least one speaker located at the inner volume in a location selected from a group consisting of upper part of the incubator, lower part of the incubator, side parts of the incubator, and any combination thereof.

According to another embodiment of the present invention, wherein the sound system provides directionality of sound.

According to another embodiment of the present invention, additionally comprising a step of playing sounds heard in the womb.

According to another embodiment of the present invention, additionally comprising a step of selecting the sounds heard in the womb from a group consisting of: heartbeat sounds, breathing sounds, gastrointestinal tract sounds, and any combination thereof.

According to another embodiment of the present invention, additionally comprising a step of muffling outside noises as heard from within the womb.

According to another embodiment of the present invention, additionally comprising a step of selecting the outside noises from a group consisting of the mother's voice, father's voice, noises of the environment of the mother, and any combination thereof.

According to another embodiment of the present invention, additionally comprising a step of changing the sound provided in a cycle of about 24 hours.

According to another embodiment of the present invention, additionally comprising a step of mimicking a daily routine of the mother; the sounds changes in a manner selected from a group consisting of: type of noise, intensity of noise, directionality of noise, and any combination thereof.

According to another embodiment of the present invention, wherein the sound system comprises at least two speakers.

According to another embodiment of the present invention, additionally comprising a step of providing different sounds by at least two speakers provide.

According to another embodiment of the present invention, additionally comprising a step of providing different strength of volume by at least two speakers provide.

According to another embodiment of the present invention, additionally comprising a step of connecting the sound system to the mother and providing live muffled sounds of the mother.

According to another embodiment of the present invention, wherein the sound system comprises a sound database.

According to another embodiment of the present invention, wherein the sound system is a learning system; the learning system is adjusting itself according to the neonate's needs.

According to another embodiment of the present invention, additionally comprising a step of adding to the bedding curves and bumps which mimics the curvatures of the womb.

According to another embodiment of the present invention, additionally comprising a step of making the bedding of materials mimicking the feeling of the womb's skin.

According to another embodiment of the present invention, wherein the bedding is a water bed; the water bed is giving the neonate the feeling of movement within water.

According to another embodiment of the present invention, additionally comprising a step of filling the bedding with a liquid of changing viscosity; the change in viscosity controls the movement of the bedding and can be accommodated to the neonate's needs.

According to another embodiment of the present invention, additionally comprising a step of changing the viscosity in a manner selected from a group consisting of: manually, as a response to an indication from the neonate, and any combination thereof.

According to another embodiment of the present invention, additionally comprising a step of selecting the indication from a group consisting of: breathing, pulse, blood pressure, sweating and any combination thereof.

According to another embodiment of the present invention, additionally comprising a step of adding to the bedding a pocket for providing pseudo-kangaroo care.

According to another embodiment of the present invention, additionally comprising a step of making the bedding from accommodating materials; the accommodating materials accommodate to at least one selected from a group consisting of: the posture of the neonate, the center of gravity of the neonate, and any combination thereof.

According to another embodiment of the present invention, wherein additionally comprising a step of moving the incubator by the movement system in at least one manner selected from a group consisting of: rotational movement, vertical movement, horizontal movement, vibrational movement, and any combination thereof.

According to another embodiment of the present invention, additionally comprising a step of controlling the movement provided by the movement system by at least one manner selected from a group consisting of speed, directionality, type of movement, degree of movement, and any combination thereof.

According to another embodiment of the present invention, additionally comprising a step of changing the movement provided by the movement system changes in a cycle of about 24 hours.

According to another embodiment of the present invention, additionally comprising a step of mimicking a daily routine of the mother; the movement changes in a manner selected from a group consisting of: type of movement, intensity of movement, and any combination thereof.

According to another embodiment of the present invention, additionally comprising a step of altering by the PECS at least one specific parameter in the surrounding environment to mimic the inner-womb environment in the inner volume.

According to another embodiment of the present invention, wherein the PECS comprises at least one filter for screening out from the surrounding environment at least one selected from a group consisting of: motion intensity, light intensity, light spectrum, sound intensity, sound spectrum, and any combination thereof.

According to another embodiment of the present invention, additionally comprising a step of integrating the filter within at least part of the envelope.

According to another embodiment of the present invention, additionally comprising a step of adjusting the filter.

According to another embodiment of the present invention, additionally comprising a step of reducing by at least one filter light penetration from the surrounding environment.

According to another embodiment of the present invention, additionally comprising a step of reducing by at least one filter light penetration to about X % of the surrounding environment's light.

According to another embodiment of the present invention, wherein X equals about 0.1% to about 1%.

According to another embodiment of the present invention, additionally comprising a step of providing light in a specific spectrum.

The method of 63, additionally comprising a step of providing light in the red spectrum.

According to another embodiment of the present invention, additionally comprising a step of reducing sounds from the surrounding environment.

According to another embodiment of the present invention, additionally comprising a step of selecting the PECS from a group consisting of sound filter, sound muffler, sound softener, and any combination thereof.

According to another embodiment of the present invention, additionally comprising a step of enabling penetration of sound in a specific frequency spectrum.

According to another embodiment of the present invention, additionally comprising a step of enabling penetration of low frequency sound into the inner volume.

According to another embodiment of the present invention, additionally comprising a step of enabling penetration of vowel sounds into the inner volume and screens out consonant sounds.

According to another embodiment of the present invention, wherein the PECS comprise a shock absorber; the shock absorber is reducing about 100% of movement resulting from the surrounding environment.

According to another embodiment of the present invention, additionally comprising a step of attaching to the incubator a feedback system for determining the neonate's level of stress.

According to another embodiment of the present invention, additionally comprising a step of determining the level of stress according to at least one parameter selected from a group consisting of: pulse, blood pressure, breathing pattern, electrical resistance, sweating and any combination thereof.

According to another embodiment of the present invention, additionally comprising a step of activating the ECS in response to the feedback system.

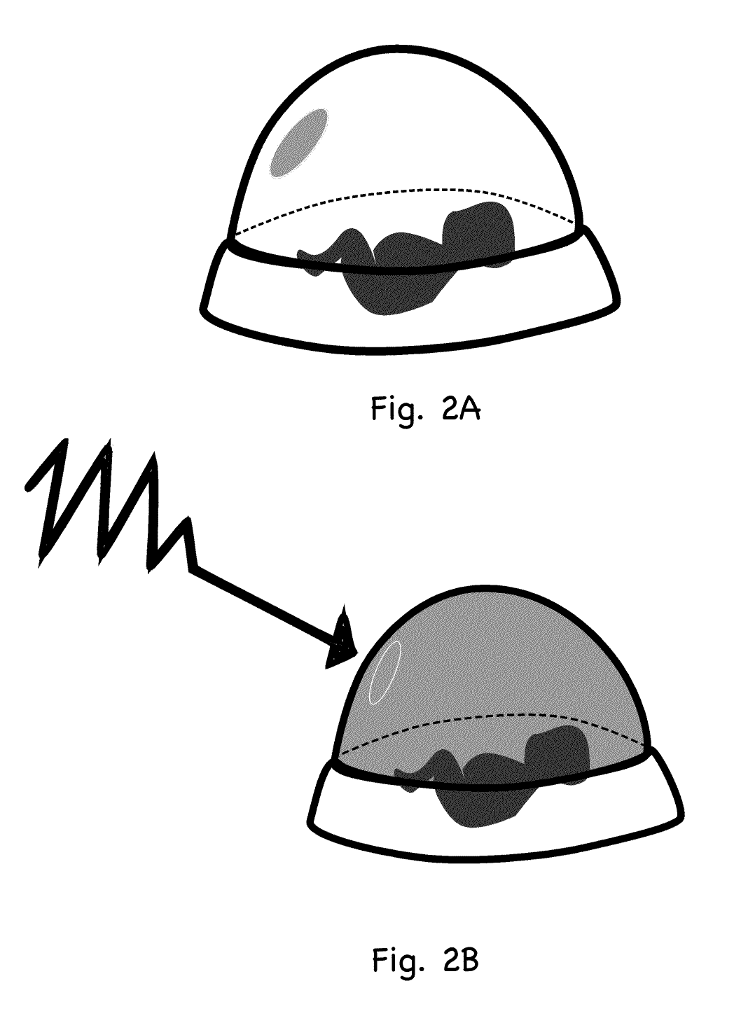

Reference is now made to FIG. 2A and FIG. 2B. FIG. 2A is a schematic illustration of incubator having no light filters. Therefore the defined environment within the incubator and the outer environment are identical regarding the light. The light inside is similar to the light outside. On the other hand, FIG. 2B shows an incubator with a light filter. The light filter is a passive environment changing system (PECS). It may either block completely the light and then a passive ECS (AECS) is needed to create a new light environment. The PECS can also stand by itself and only filter out the light. Filtration can be in manner of intensity or light spectrum. For example, the light filter can screen only light in the red spectrum.

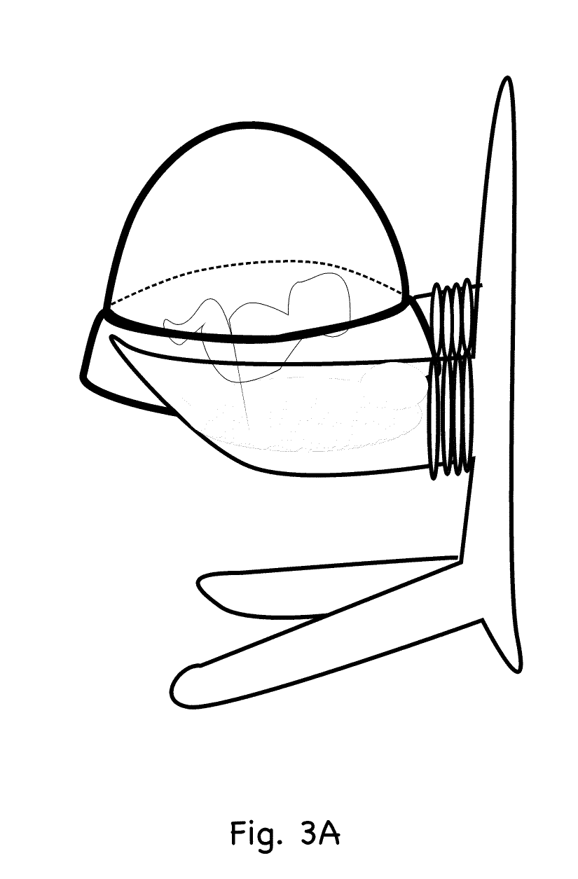

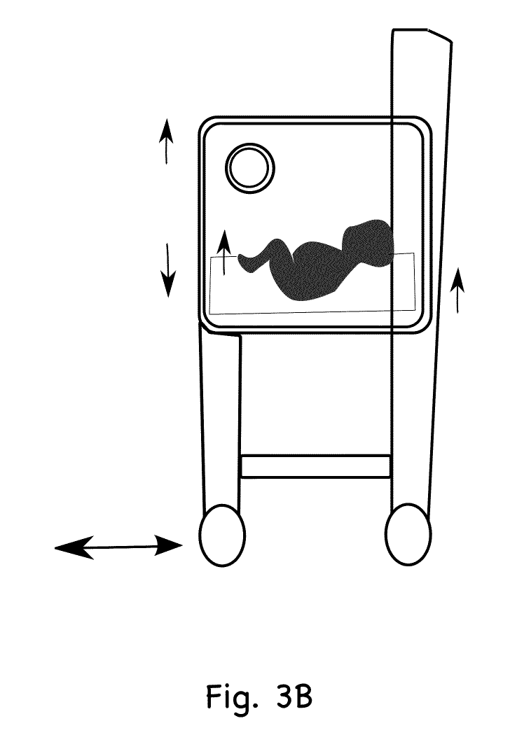

Reference is now made to FIGS. 3A and 3B which are illustrations of preferred embodiments of a neonate's incubator having different movement systems. FIG. 3A shows an incubator held by two arms connected to motors. The motor can move the arms in several directions and create a pattern of movement. The movement can be manually or automatically adjusted in manner concerned with directionality as well as speed and intensity. FIG. 3b illustrates an incubator having several levels of movements. The bedding can move in on direction, the incubator in a second direction and the cart holding the incubator in a third direction thus creating together an orchestrated movement that can be calibrated by changing each of these components.

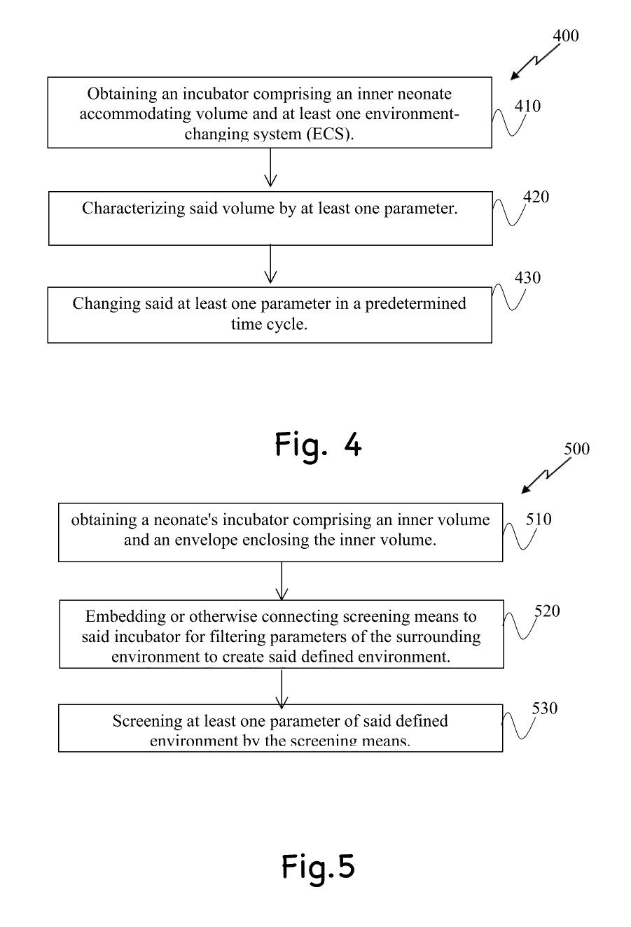

FIG. 4 is a schematic flow diagram describing another preferred method for changing a defined environment within a neonate's incubator to mimic the inner-womb environment (400). In the first step (410) an incubator is obtained comprising an inner neonate accommodating volume and at least one environment-changing system (ECS). In the next step, at least one parameter is characterizing (420). Finally at least one parameter is changed in a predetermined time cycle (430). The ECS may be passive or active.

FIG. 5 is a schematic flow diagram describing a preferred method for changing a defined environment within a neonate's incubator to mimic the inner-womb environment (500). This method includes obtaining an incubator (510) and embedding to it screening means (520) for filtering parameters from the surrounding environment to create a defined environment within the incubator (530). The screening means may be embedded to at least part of the envelope to the incubator.

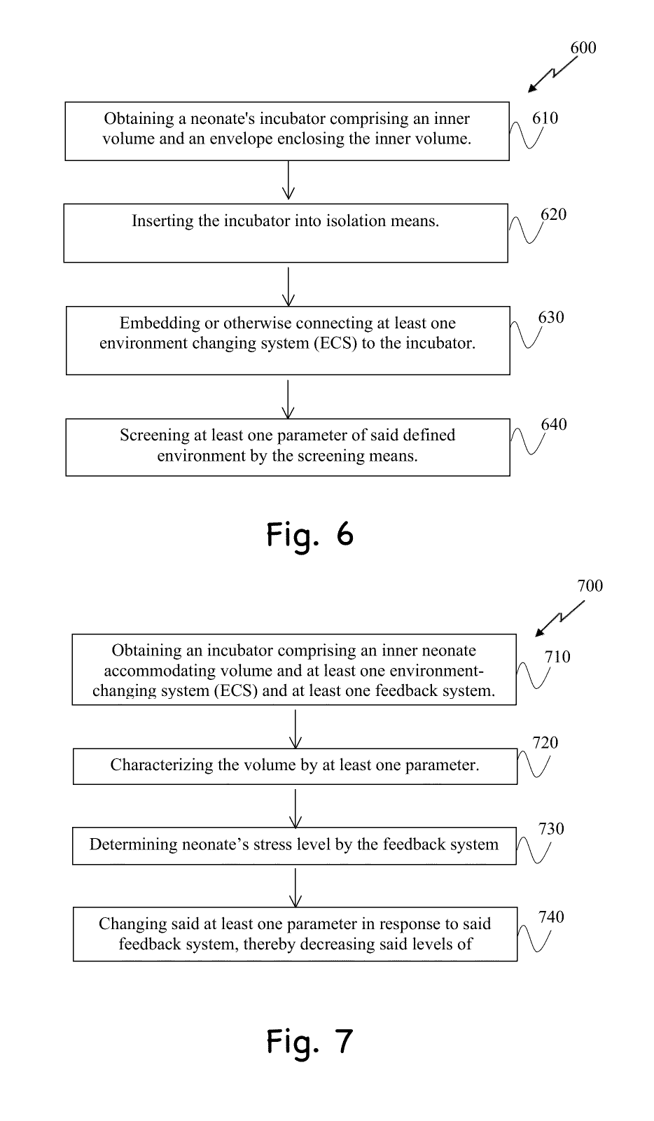

FIG. 6 is a schematic flow diagram describing another preferred method for changing a defined environment within a neonate's incubator to mimic the inner-womb environment (600). This method includes obtaining an incubator (610) and inserting the incubator into isolating means (620) and embedding to it an ECS (630). The same parameter is isolated from the surrounding and artificially creates it to form a womb mimicking environment.

FIG. 7 is a schematic flow diagram describing a preferred method for changing a defined environment within a neonate's to reduce stress of the neonate (700). This method includes obtaining an incubator comprising an inner neonate accommodating volume and at least one environment-changing system (ECS) and at least one feedback system (710) and characterizing the volume by at least one parameter (720). Determining neonate's stress level by the feedback system (730) and changing said at least one parameter in response to said feedback system (740), thereby decreasing said levels of neonate's stress. The ECS is activated as a response to the feedback system and tries to reduce the stress level as much as possible.

1. A womb mimicking neonate's incubator, wherein said incubator comprises an inner neonate accommodating volume and at least one environment-changing system (ECS); said volume is characterized by at least one parameter; said ECS is configured to change said at least one parameter in a predetermined time cycle which mimics human inner-womb environment.

2. The neonate's incubator of claim 1, wherein said at least one parameter is selected from a group consisting of spectrum of light, intensity of light, scattering of light, directionality of light, light's daily profile; spectrum of sound, amplitude of sounds, intensity of sound, directionality of sound, type of sound, sound's daily profile, rhythm of motion, intensity of motion, directionality of motion, motion's daily profile, scent, sensation, humidity, temperature and any combination thereof.

3. The neonate's incubator of claim 1, wherein said ECS is selected from a group consisting of passive ECS (PECS), active ECS (AECS), and any combination thereof; said AECS is selected from a group consisting of at least one light system, at least one sound system, at least one scent creator, at least one movement system, bedding and any combination thereof; said PECS blocks or filters from said surrounding environment at least one selected from a group consisting of motion, light, sound, scent and any combination thereof.

4. The neonate's incubator of claim 3, wherein said incubator comprises at least one PECS blocking said at least one specific parameter and at least one AECS artificially creating said at least one specific parameter.

5. The neonate's incubator of claim 1, wherein said incubator additionally comprises a feedback system for determining said neonate's level of stress; said level of stress is determined according to at least one factor selected from a group consisting of: pulse, blood pressure, breathing pattern, electrical resistance, sweating and any combination thereof; said ECS activity changes in response to said feedback system.

6. The neonate's incubator of claim 1, wherein said cycle is the cycle of the mother of said neonate while carrying him in her womb.

7. A womb mimicking neonate's incubator, wherein said incubator comprises an inner neonate accommodating volume, at least one environment-changing system (ECS) and at least one feedback system; said volume is characterized by at least one parameter; said feedback system determines said neonate's stress level; said ECS is adapted to change said at least one parameter in response to said feedback system to decrease said levels of stress.

8. The neonate's incubator of claim 7, wherein said at least one parameter is selected from a group consisting of spectrum of light, intensity of light, scattering of light, directionality of light, light's daily profile; spectrum of sound, amplitude of sounds, intensity of sound, directionality of sound, type of sound, sound's daily profile, rhythm of motion, intensity of motion, directionality of motion, motion's daily profile, scent, sensation, humidity, temperature and any combination thereof.

9. The neonate's incubator of claim 7, wherein said ECS is selected from a group consisting of passive ECS (PECS), active ECS (AECS), and any combination thereof; said AECS is selected from a group consisting of: at least one light system, at least one sound system, at least one scent creator, at least one movement system, bedding and any combination thereof; said PECS blocks or filters from said surrounding environment at least one selected from a group consisting of: motion, light, sound, scent and any combination thereof.

10. The neonate's incubator of claim 9, wherein said incubator comprises at least one PECS blocking said at least one parameter and at least one AECS artificially creating said at least one parameter.

11. The neonate's incubator of claim 7, wherein said feedback system determines said neonate's level of stress according to at least one factor selected from a group consisting of: pulse, blood pressure, breathing pattern, electrical resistance, sweating and any combination thereof.

12. The neonate's incubator of claim 9, wherein said PECS block about 100% of said at least one parameter; said at least one parameter is selected from a group consisting of: said surrounding environment's sound, said surrounding environment's light, said surrounding environment's motion, said surrounding environment's scent, and any combination thereof.

13. The neonate's incubator of claim 9, wherein said ECS accommodates said at least one parameter according to a cycle.

14. The neonate's incubator of claim 13, wherein said cycle is the cycle of the mother of said neonate while carrying him in her womb.

15. A method for mimicking inner womb environment within a neonate's incubator; said method is characterized by steps of:
a. obtaining an incubator comprising an inner neonate accommodating volume and at least one environment-changing system (ECS);
b. characterizing said volume by at least one parameter; and,
c. changing said at least one parameter in a predetermined time cycle, thereby mimicking, within said volume, said inner-womb environment.

16. The method of claim 15, additionally comprising a step of selecting said at least one parameter selected from a group consisting of spectrum of light, intensity of light, scattering of light, directionality of light, light's daily profile; spectrum of sound, amplitude of sounds, intensity of sound, directionality of sound, type of sound, sound's daily profile, rhythm of motion, intensity of motion, directionality of motion, motion's daily profile, scent, sensation, humidity, temperature and any combination thereof.

17. The method of claim 15, additionally comprising a step of selecting said ECS selected from a group consisting of passive ECS (PECS), active ECS (AECS), and any combination thereof; said AECS is selected from a group consisting of at least one light system, at least one sound system, at least one scent creator, at least one movement system, bedding and any combination thereof; said PECS blocks or filters from said surrounding environment at least one selected from a group consisting of motion, light, sound, scent and any combination thereof.

18. The method of claim 17, additionally comprising a step of blocking said at least one specific parameter by said at least one PECS and artificially creating said at least one specific parameter by said at least one AECS.

19. The method of claim 15, additionally comprising a step of determining said neonate's level of stress by a feedback system for; said level of stress is determined according to at least one factor selected from a group consisting of: pulse, blood pressure, breathing pattern, electrical resistance, sweating and any combination thereof; said ECS activity changes in response to said feedback system.

20. The method of claim 15, wherein said cycle is the cycle of the mother of said neonate while carrying him in her womb.

21. A method for mimicking inner womb environment within a neonate's incubator; said method is characterized by steps of:
a. obtaining an incubator comprising an inner neonate accommodating volume and at least one environment-changing system (ECS) and at least one feedback system;
b. characterizing said volume by at least one parameter;
c. determining said neonate's stress level by said feedback system; and,
d. changing said at least one parameter in response to said feedback system, thereby decreasing said levels of neonate's stress.

22. The method of claim 21, additionally comprising a step of selecting said at least one parameter selected from a group consisting of spectrum of light, intensity of light, scattering of light, directionality of light, light's daily profile; spectrum of sound, amplitude of sounds, intensity of sound, directionality of sound, type of sound, sound's daily profile, rhythm of motion, intensity of motion, directionality of motion, motion's daily profile, scent, sensation, humidity, temperature and any combination thereof.

23. The method of claim 21, additionally comprising a step of selecting said ECS selected from a group consisting of passive ECS (PECS), active ECS (AECS), and any combination thereof; said AECS is selected from a group consisting of at least one light system, at least one sound system, at least one scent creator, at least one movement system, bedding and any combination thereof; said PECS blocks or filters from said surrounding environment at least one selected from a group consisting of motion, light, sound, scent and any combination thereof.

24. The method of claim 23, additionally comprising a step of blocking said at least one specific parameter by said at least one PECS and artificially creating said at least one specific parameter by said at least one AECS.

25. The method of claim 21, additionally comprising a step of determining said neonate's level of stress according to at least one factor selected from a group consisting of: pulse, blood pressure, breathing pattern, electrical resistance, sweating and any combination thereof.

26. The method of claim 23, additionally comprising a step of blocking by said PECS about 100% of a said at least one parameter; said at least one parameter is selected from a group consisting of: said surrounding environment's sound, said surrounding environment's light, said surrounding environment's motion, said surrounding environment's scent, and any combination thereof.

27. The method of claim 23, additionally comprising a step of accommodating by said ECS said at least one parameter according to a cycle.

28. The method of claim 27, wherein said cycle is the cycle of the mother of said neonate while carrying him in her womb.
